(19) United States
(12) Patent Application Publication
(10) Pub. No.: US 2014/0271463 A1
(43) Pub. Date: Sep. 18, 2014

(54) BRI2 AS A NOVEL BIOMARKER FOR ALZHEIMER'S DISEASE

(71) Applicant: Stichting VU-VUmc, Amsterdam (NL)

(72) Inventors: Marta DEL CAMPO MILAN, Amsterdam (NL); Cornelia Ramona Jimenez, Amsterdam (NL); Chrlott Elisabeth Teunissen, Amsterdam (NL)

(73) Assignee: Stichting VU-VUmc, Amsterdam (NL)

(21) Appl. No.: 14/207,396

(22) Filed: Mar. 12, 2014

Related U.S. Application Data
(60) Provisional application No. 61/776,938, filed on Mar. 12, 2013.

(51) Int. Cl. C07K 16/28; A61K 51/10; G01N 33/569

(52) U.S. Cl. CPC C07K 16/28; G01N 33/56966; A61K 51/1027
USPC 424/1.49; 435/7.21; 424/172.1; 424/139.1; 530/387.9; 536/23.53; 435/320.1; 436/501

(57) ABSTRACT

The disclosure relates to the use of altered BRI2 levels as a biomarker for the risk of developing Alzheimer's disease. Novel treatments based on altered BRI2 levels and anti-BRI2 antibodies are also provided.

### CROSS-REFERENCE TO RELATED APPLICATION

This application claims the benefit under 35 U.S.C. §119(e) to U.S. Patent Application Ser. No. 61/776,938 filed on Mar. 12, 2013, the contents of the entirety of which are incorporated herein by this reference.

### TECHNICAL FIELD

The disclosure relates to biotechnology and the use of altered BRI2 levels as a biomarker for the risk of developing Alzheimer's disease. Novel treatments bases on altered BRI2 levels and anti-BRI2 antibodies are also provided.

### BACKGROUND OF THE INVENTION

Alzheimer's disease (AD) is an age-related irreversible neurodegenerative disorder and the most common form of dementia. The main pathological hallmarks of AD are the presence of neurofibrillary tangles (NFTs) constituted by the phosphorylated protein tau (P-tau) and the accumulation of amyloid β (Aβ) peptide, which leads to the development of amyloid plaques. Although the aetiology of AD remains unknown, the main theory to date about AD pathogenesis is the amyloid cascade hypothesis, which suggests that Aβ accumulation is the key event leading to neuronal loss. However, more than 90% of AD cases are sporadic. Moreover, the deposition of Aβ in amyloid plaques is also observed in normal aging and its correlation with neuronal loss and cognitive decline is not strong. These data have brought many scientists to suggest another alternative hypothesis stating that Aβ plaque and NFT formation might be disease bystanders rather than initiating events of the disease (Lee H et al. (2007) Amyloid in Alzheimer Disease: The Null versus the Alternate Hypotheses. 321:823-829.).

Early treatment of Alzheimer's disease could slow or delay the progression of the disorder leading to an improved quality of life. The inability to point to a definitive cause for most cases of Alzheimer's disease, however, makes early detection difficult. Further insight into the causes of cognitive decline in AD is thus necessary in order to develop effective treatments and methods for early detection.

### SUMMARY OF THE DISCLOSURE

One aspect of the disclosure provides a method for determining the level of BRI2 polypeptide in an individual, the method comprising contacting a sample from the individual with a BRI2 binding compound. Preferably, the contacting occurs in vitro.

One aspect of the disclosure provides a method of determining the risk of developing Alzheimer's disease in an individual, comprising determining the level of BRI2 polypeptide in the individual and comparing the level of BRI2 to a reference value.

Preferably, a BRI2 level higher than the reference value indicates a risk of developing Alzheimer's disease.

Preferably, in the disclosed methods the level of BRI2 in determined in vitro from a sample from the individual. Preferably, the sample is cerebral spinal fluid or blood.

Preferably, the level of BRI2 is determined with a BRI2 binding compound. Preferably, the binding compound binds to amino acids 137-231 of SEQ ID NO:1.

Preferably, the binding compound binds to amino acids 140-153 of SEQ ID NO:1.

Preferably, wherein the binding compound is a monoclonal antibody.

Preferably, the binding compound is a polyclonal antibody, which does not bind to SEQ ID NO:1 at amino acids outside of 140-153.

Preferably, the level of BRI2 is determined in vivo. Preferably, the level of BRI2 is determined in the hippocampus of the individual.

Provided is a method comprising: (a) administering to an individual a positron emission tomography (PET)-compatible tracer which binds to BRI2; (b) carrying out a PET scan of the individual; and (c) determining the signal intensity of the tracer. Preferably, a tracer intensity higher than a reference value indicates a risk of developing Alzheimer's disease.

One aspect of the disclosure provides a method for identification of compounds for the treatment of a Alzheimer's disease during preclinical stages, the method comprising: (a) administering one or more candidate compounds to a preclinical animal model of Alzheimer's disease; (b) assessing changes in BRI2 in the animal model relative to measures of BRI2 in a control animal; and (c) selecting a candidate compound that induces a change in BRI2 toward measures of BRI2 in a control animal.

One aspect of the disclosure provides a method for treating Alzheimer's disease in an individual, comprising administering to an individual in need thereof a therapeutically effective amount of a compound, which reduces the level of BRI2 protein. Preferably, the compound is a BRI2 binding molecule, preferably an antibody. Preferably, the binding molecule binds to amino acids 137-231 of SEQ ID NO:1. Preferably, the binding molecule binds to amino acids 140-153 of SEQ ID NO:1.

Preferably, the compound reduces the level of abnormal or non-functional BRI2 protein.

One aspect of the disclosure provides an anti-BRI2 antibody that binds to amino acids 140-153 of SEQ ID NO:1. Preferably, the antibody is a monoclonal antibody. Preferably, the antibody is polyclonal and does not bind to SEQ ID NO:1 at amino acids outside of 140-153.

One aspect of the disclosure provides a nucleic acid molecule encoding an antibody disclosed herein.

One aspect of the disclosure provides a vector comprising at least one nucleic acid molecule disclosed herein.

One aspect of the disclosure provides the use of the antibody, disclosed herein, for determining the risk of developing Alzheimer's disease in an individual.

One aspect of the disclosure provides a method for treating Alzheimer's disease in an individual, comprising:

a) determining the level of BRI2 polypeptide in the individual,
b) comparing the level of BRI2 to a reference value; and
c) treating an individual having an altered level of BRI2 over the reference value with an Alzheimer's disease treatment, preferably selected from an acetyl cholinesterase inhibitor, memantine, or an NMDA receptor antagonist.

### BRIEF DESCRIPTION OF THE DRAWINGS

FIG. 1: Antibodies raised against human BRI2 were specific on Western Blot.

A, Schematic illustration of the BRI2 protein. The cell membrane, the BRICHOS domain and BRI223 peptide are presented. The numbers (1, 2 and 3) indicate the cleavage sites of BRI2 for different enzymes (furin, ADAM10 and SPPL2b, respectively). The enlargement of the BRICHOS domain shows the aa sequence (amino acids 137-231 of SEQ ID NO:1) that is recognized by BRI2140-153 antibody. B, Proteomic analysis of CSF samples from SMC (n=4), MCI-S (n=4), MCI-AD (n=5) and AD (n=5) cases. Higher spectral counts of BRI2 peptides were found in MCI-AD and AD compared to SMC and MCI-S. C-F: Western blot analysis of human brain homogenates from AD and Control (Cn) cases showed reactivity at bands of 40, 45 and 52 kDa using polyclonal antibodies raised against human BRI2140-153 (C, affinity-purified and D, protein A-purified). E, Pre-absorption of polyclonal protein A-purified BRI2140-153 antibody with BRI2140-153 peptide dramatically diminished or even abrogated the signal of all the bands. F, Similar reactivity against AD and Control cases was observed using the monoclonal IgM-purified anti-BRI2111-153. G, Several bands at different molecular weights were observed when recombinant BRI276-266 (Bri) was analyzed using protein A-purified anti-BRI2140-153 indicating that BRI276-266 can form aggregates of various sizes.

FIG. 2: BRI2 is increased in human brain homogenates of AD patients compared to controls. A, Representative Western blot of HBH from 3 Control and 3 AD cases indicating an increase of BRI2 in AD. Actin analysis showed equal protein concentrations in every lane. B, BRI2 reactivity against human brain homogenates from control (n=14) and AD (n=14) patients was quantified and corrected for actin levels. Data were compared based on pathological diagnosis. +: Cases that neuropathologically could not be classified as either AD or control (uncertain, n=3), but were clinically diagnosed as AD.

FIG. 3: BRI2 deposition in AD hippocampus is associated with amyloid plaques. A, Schematic representation of human hippocampus. Arrows delimit the different areas analyzed: CA4-2, CA1 and Subiculum. B, BRI2 deposition in AD post-mortem hippocampus, visualized with anti-BRI140-153 (Red). C, Post-mortem hippocampus sections from control and AD cases were stained with anti-BRI140-153 (Red). AD cases were also simultaneously stained for anti-BRI140-153 (Red) and anti-Aβ1-17 (Brown). BRI2 deposition in plaques is present (arrows with numbers) in all AD brain areas, but not in controls. Double immunohistochemistry showed that BRI2 deposition was associated with amyloid plaques. Scale bars: 100 μm.

FIG. 4: BRI2 immunoreactivity is significantly more frequent in AD cases. BRI2 immunoreactivity (IR) was semiquantitatively measured for each patient in each hippocampus area, and patients were grouped according to pathological diagnosis. Higher numbers of BRI2 deposits were found in AD patients (n=14) compared to control (n=14) cases in all analyzed areas. +: Cases that neuropathologically could not be classified as either AD or control (uncertain, n=3), but were clinically diagnosed as AD.

FIG. 5: BRI2 deposition starts in early stages of the disease. Analysis of BRI2 immunoreactivity (IR) in human hippocampus according to Braak stage for NFTs (A) or Thal staging for amyloid pathology (B). BRI2 was increased already in early stages of the disease (Braak III-2/3).

FIG. 6: The levels of the BRI2 processing enzymes furin, ADAM10 and SPPL2b are changed in AD human hippocampus. A, Representative Western blot of human brain homogenates from 3 control and 2 AD patients shows the reactivity of furin, ADAM10, SPPL2b and BRI2 in the same samples. Actin analysis showed equal protein concentrations in every lane. B-D: Significant differences between Control (n=14) and AD (n=14) patients are observed in the levels of ADAM10 (C) and SPPL2b (D) and a tendency for the levels of furin (B). E-G: Correlation analysis showed a positive correlation between the levels of furin and ADAM10 (E), an inverse correlation between ADAM10 and BRI2 (F); and no significant correlation between furin and BRI2 (G).

FIG. 7: BRI2-APP complexes are present in control but not in AD human hippocampus. A, BRI2 was immunopurified from human hippocampus of 2 controls and 2 AD cases using anti-BRI2113-231. APP was analyzed by Western blot in the original samples (untreated) and in the immunopurified-BRI2 samples (BRI2 IP). Braak stages are shown on top of the blots. Negative control (−Ctr) is an IP performed with an irrelevant rabbit antibody. B, Immunopurification of BRI2 was repeated in two other controls cases and two AD patients with a slightly different method. APP was analyzed by Western blot in the immunopurified-BRI2 samples (BRI2 IP). Braak stages are shown on top of the blots. Negative controls are IPs performed without sample (no sample) and an IP performed with BRI2 pre-absorbed antibody (Preabs. BRI2).

FIG. 8: Hypothetical model illustrating the possible causes and consequences of BRI2 deposition. Since the levels of furin and ADAM10 were significantly correlated and furin positively regulates ADAM10 (Hwang et al. 2006), reduced levels of furin may lead to a reduced levels of ADAM10. The lack of ADAM10 cleavage and further processing of SPPL2b leads to the release of the whole BRI2 ectodomain. BRI2 ectodomain has high aggregation propensities and thus, its release may lead to the observed accumulation and deposition of BRI2. Altogether, it would prevent the formation of BRI2-APP complexes and the subsequent inhibition of Aβ production and aggregation.

FIG. 9: Antibodies raised against human BRI2 show that the observed 45 kDa band in Western blot and the BRI2 deposition in immunohistochemistry are specific. A, The 45 kDa BRI2 band observed by western blot with the polyclonal anti BRI2140-153 was specific for BRI2 as several antibodies raised against the BRICHOS domain (monoclonal protein G-purified anti-BRI2111-153, monoclonal protein G-purified BRI2140-153 and polyclonal protein G-purified BRI2113-231) show a similar reactivity for AD and controls (Cn) human hippocampus homogenates, and the signal disappeared after pre-absorbing each antibody (Preabs.) with BRI276-266 (for anti-BRI2111-153 and BRI2113-231) or BRI2140-153 (for monoclonal anti-BRI2140-153). B, Several bands at different molecular weights were observed when recombinant BRI276-266 (Bri) was analyzed using protein G-purified goat anti BRI2113-231 indicating that BRI276-266 can form aggregates of various sizes. C-F, A similar BRI2 deposition pattern in AD plaques was observed for all antibodies. C) polyclonal protein A-purified BRI2140-153, D) monoclonal protein G-purified BRI2140-153, E) monoclonal protein G-purified anti-BRI2111-153 and F) polyclonal goat protein G-purified BRI2113-231. G, An additional double staining using polyclonal protein A-purified BRI2140-153 and mouse monoclonal Aβ1-17 is shown to observe the association with an amyloid plaque. H, Pre-absorption of Protein A-purified BRI2140-153 with its antigenic peptide completely abrogates the BRI2 reactivity observed by double immunohistochemistry analysis with monoclonal Aβ31-17 in AD hippocampus tissue.

FIG. 10: BRI2 is increased in human brain homogenates of AD patients compared to controls. BRI2 reactivity against human brain homogenates from control (n=14) and AD patients (n=15) was measured and corrected for actin levels. Data were compared based on clinical diagnosis. Two patients clinically diagnosed with vascular dementias were pathologically diagnosed as AD and its BRI2 levels were within the range of the AD group.

FIG. 11: Approximately 50% of Aβ plaques are associated with BRI2 in human hippocampus. A, The total amount of Aβ plaques was counted and the percentage of BRI2 positive Aβ plaques was calculated in CA4-2, CA1 and Subiculum in AD and control human hippocampus sections. Approximately 50% of Aβ plaques are BRI2 positive. Only cases with Aβ plaques were analyzed, including control cases that developed a low number of Aβ plaques.

FIG. 12: BRI2 deposition is significantly higher in AD cases. BRI2 immunoreactivity (IR) was quantified for each patient in each hippocampus area and levels were compared with clinical diagnosis as outcome measure. AD patients (n=12) showed higher BRI2 deposition compared to control cases (n=18) in all analyzed areas. All the patients that were clinically diagnosed as vascular dementia (n=3) were pathologically diagnosed as AD and had a BRI2 reactivity according to the AD group.

FIG. 13: BRI2 45 kDa increased correlates with the BRI2 deposition observed in all areas of human AD hippocampus. BRI2 immunoreactivity (IR) was quantified in each hippocampus area and correlated to the levels of BRI2 45 kDa for each patient (n=29). There was a significant correlation between the levels of 45 kDa BRI2 band in Western blot and the immunohistochemistry results in every hippocampal area.

FIG. 14: The levels of SPPL2b did not correlate with the levels of BRI2, ADAM10 and furin in human hippocampus. The levels of BRI2, furin, ADAM10 and SPPL2b were analyzed in the same hippocampus homogenates from different patients (n=30). No significant correlations were observed between SPP12b and the different proteins analyzed.

FIGS. 15A and 15B: BRI2 reduction in CSF of AD patients. 15A) Reduced BRI2 fluorescence intensity in CSF of patients with a typical biochemical AD profile compared to memory clinic patients with a typical non-AD (control) profile; P<0.021. 15B) Reduction in signal by 60% after pre-incubation with the BRI2 protein shows specificity of the signal.

### DETAILED DESCRIPTION OF THE DISCLOSED EMBODIMENTS

The disclosure is based, in part, on the finding that BRI2 protein levels are altered in hippocampus tissue homogenates from Alzheimer's disease patients. In particular, alterations were identified in the early stages of the pathology.

BRI2 (also known as Itm2B) is a type II transmembrane protein with unknown physiological function. During maturation, BRI2 can be cleaved by a furin-like protease in its C-terminal region which leads to the release of a 23 amino acid peptide (ABri₂₃) (Kim S H et al. (1999) Nature neuroscience 2:984-8). The remaining membrane-bound N-terminal part of BRI2 (mBRI2) contains a BRICHOS domain and can be further processed by ADAM10 and SPPL2b (Martin L et al. (2008) Regulated intramembrane proteolysis of Bri2 (Itm2b) by ADAM10 and SPPL2a/SPPL2b. The Journal of biological chemistry 283:1644-52). Processing of mBRI2 by the α-secretase ADAM10 leads to the secretion of a 25 kDa peptide containing the BRICHOS domain. The remaining membrane N-terminal fragment (NTF) of BRI2 undergoes an additional proteolysis by SPPL2a/b leading to the release of a small-secreted C-peptide and the liberation of a 10 kDa intracellular domain (ICD) in the cytosol (see FIG. 1).

Recent studies revealed a possible link between BRI2 and the main proteins involved in AD pathogenesis, e.g., APP and abeta(1-42). Both ABri₂₃ and the BRICHOS domain of BRI2 have been shown to inhibit Aβ₄₂ aggregation in vitro and in vivo (Peng S, Fitzen M, Jörnvall H, Johansson J (2010) Biochemical and biophysical research communications 393:356-6). Moreover, mBRI2 is able to bind amyloid 13 precursor protein (APP), leading to decreased production of Aβ₄₀ and Aβ₄₂ in both transgenic mice and cell cultures (Fotinopoulou A et al. (2005) The Journal of biological chemistry 280:30768-72). However, there is no evidence that alterations in BRI2 levels or activity are a causal factor for AD.

Familial British dementia (FBD) and familial Danish dementia (FDD) are two early-onset autosomal dominant disorders caused by mutations in the BRI2 gene. In FBD, a point mutation at the stop codon enlarges the reading frame of the cDNA resulting in the production of a longer BRI2 precursor protein (ABriPP) of 277 amino acids (Vidal R et al. (1999) A stop-codon mutation in the BRI gene associated with familial British dementia. Nature 399:776-81). In FDD cases, an elongated mutated protein of 277 amino acids (ADanPP) is also generated due to a decamer duplication insertion between codons 265 and 266 of the wild-type BRI2 (wtBRI2) cDNA (Vidal R et al. (2000) Proceedings of the National Academy of Sciences of the United States of America 97:4920-5). Both mutations lead to a 34 amino acids C-terminal sequence of ABriPP and ADanPP of which the first 22 amino acids are identical to that of wild-type BRI2. However, the 12 additional amino acid C-terminal segment within the mutated proteins are completely different from each other. Similar to the wtBRI2, ABriPP and ADanPP also undergo the same furin-like proteolytic processing at positions 243-244 leading to the secretion of 34 amino acid peptides named British amyloid (ABri) and Danish amyloid (ADan) (Kim S H et al. (1999) Furin mediates enhanced production of fibrillogenic ABri peptides in familial British dementia. Nature neuroscience 2:984-8). These peptides were isolated from amyloid deposits in FBD and FDD cases, respectively, (Vidal R et al. 1999; Vidal R et al. 2000). It has been shown that both ABri and ADan have a high tendency to aggregate and oligomerize in vitro and in vivo (Rostagno a et al. (2005) Chromosome 13 dementias. Cellular and molecular life sciences:CMLS 62:1814-25.). These results suggest that specific mutations in the C-terminal segment of BRI2 are linked to neurodegenerative/dementia pathology.

Examples 2 and 3, described herein, demonstrate that BRI2 levels are increased in the hippocampus of AD patients compared to controls and that BRI2 accumulates in AD hippocampus in early pathological stages and associates with amyloid plaques. In further studies, we have demonstrated that BRI2 levels are decreased in the cerebral spinal fluid (CSF) of AD patients compared to controls (see FIG. 15A).

As already described, BRI2 is processed into various polypeptide fragments, such as BRI2₂₃, an N-terminal fragment, and a BRICHOS containing domain. While mutations in BRI2₂₃ have been associated with FBD and FDD, the disclosure provides that alterations in the levels of the BRI2 BRICHOS containing domain are associated with an increased risk for Alzheimer's disease. Antibodies which recognize this domain, in particular amino acids 140-153 of human BRI2 are especially useful for diagnosing AD.

While not wishing to be bound by theory, it may be necessary to maintain certain levels of BRI2. Alterations in BRI2 protein levels (either an increase or decrease) are associated with neurodegenerative pathology. Alternatively, low levels of functional or “normal” BRI2 may be associated with AD pathology. As used herein, non-functional or abnormal BRI2 refers to BRI2 protein having a primary, secondary, or tertiary amino acid structure which differs from wild-type BRI2 such that the function of BRI2 is reduced or a new function or effect arises (e.g., overexpression or conformational change of BRI2 which leads to BRI2 aggregation, which in turn promotes the aggregation of other proteins, such as Aβ accumulation). Such abnormal BRI2 includes BRI2 with modifications, abnormal glycosylation, and dimer formation.

The BRI2 increase and deposition in AD patients observed in this study was unexpected since BRI2 has positive anti-amyloidogenic effects (Fotinopoulou et al. 2005; Matsuda et al. 2005; Peng et al. 2010; Willander et al. 2012) and its overexpression can halt AD pathology (Kim et al. 2000; Matsuda et al. 2008; Kilger et al. 2011). Moreover, BRI2 is an important protein preserving memory and cognition (Tamayev et al. 2010a, 2010b). Thus, the increased of the 45 kDa BRI2 form in AD likely reflects changes on BRI2 protein, which may affect its positive functioning. FIG. 8 represents a hypothetical model of the causes and consequences of increased levels of the 45 kDa BRI2 form in AD. In this model, reduced furin levels may lead to reduced levels of ADAM10 which, in turn, could prevent the cleavage of BRI2 that leads to the secretion of a 25 kDa peptide (Martin et al. 2008). Since BRI2 is probably further processed by SPPL2b, which is elevated in AD, the reduced levels of ADAM10 lead to higher levels of secreted BRI2 ectodomain, which is able to aggregate and thus, may promote BRI2 deposition. Based on our results, we hypothesize that BRI2 deposition may prevent its binding to APP, thereby enhancing APP processing. Additionally, the non-amyloidogenic pathway of APP processing might be also hampered in AD due to the decreased expression of ADAM10, which is the major α-secretase involved in the non-amyloidogenic APP shedding (Endres & Fahrenholz, 2012; Kuhn et al. 2010). Alterations in both pathways could increase the production and aggregation of Aβ42, ultimately resulting in amyloid plaque formation (Peng et al. 2010; Kim et al. 2008; Kuhn et al. 2010). Regardless of the mechanism, the disclosure demonstrates that alterations in BRI2 protein levels can serve as a biomarker for the risk of developing AD.

In one aspect, provided are methods for determining the level of a BRI2 polypeptide in an individual comprising contacting/analysing a sample from the individual with a BRI2 binding compound. Preferably, the contacting occurs in vivo. Preferably, the contacting occurs in vitro. Suitable in vivo and in vitro assays are described further herein. As used herein, “an individual” is any mammal including humans; laboratory animals such as rats, mice, simians and guinea pigs; domestic animals such as rabbits, cattle, sheep, goats, cats, dogs, horses, and pigs and the like. Preferably, the individual is human.

The levels of BRI2 polypeptide can provide diagnostic information regarding the risk of an individual developing Alzheimer's disease. The disclosure, thus, also provides methods of determining the risk of developing Alzheimer's disease in an individual, comprising determining the level of BRI2 protein in an individual, preferably the level of BRI2 protein is determined using a BRI2 binding compound.

As used herein, a “BRI2” polypeptide refers to, e.g., polypeptides as set forth in GenBank gi accession numbers 6680502 (mouse), NP_{—}068839.1 (human) and 55741681 (rat). A BRI2 polypeptide also includes polypeptide fragments of BRI2 having at least 10, preferably at least 20 amino acids. A human BRI2 protein sequence is as follows:

MVKVTFNSAL AQKEAKKDEP KSGEEALIIP PDAVAVDCKD PDDVVPVGQR RAWCWCMCFG LAFMLAGVIL GGAYLYKYFA LQPDDVYYCG IKYIKDDVIL NEPSADAPAA LYQTIEENIKIFEEEEVEFI SVPVPEFADS DPANIVHDFN KKLTAYLDLN LDKCYVIPLN TSIVMPPRNL LELLINIKAG TYLPQSYLIH EHMVITDRIE NIDHLGFFIY RLCHDKETYK LQRRETIKGI QKREASNCFA IRHFENKFAV ETLICS SEQ ID NO:1.

Human BRI2 comprises a cytosolic N-terminal domain of 54 aa followed by an additional 20 aa in the plasma membrane. BRI2 luminal domain (BRI276-266) contains the BRICHOS domain (aa 137-231) and a N-glycosylation site at asparagine residue 170 (Asn170).

The processing of BRI2 protein results in various polypeptide fragments. Preferably, the methods described herein, detect BRI2 polypeptides containing the BRICHOS domain.

The level of BRI2 protein in the subject can be compared to a reference value. A reference value refers to the level (amount) of a protein in a control sample (e.g., from the same type of tissue as the tested tissue, such as blood or serum, urine, saliva), from a “normal” healthy subject that does not suffer from AD. If desired, a pool or population of the same tissues from normal subjects can be used, and the reference value can be an average or mean of the measurements.

An alteration in the level of BRI2 protein in an individual as compared to the reference value indicates the risk of developing AD. In preferred embodiments, a decrease in the level of BRI2 protein as compared to the reference value indicates the risk of developing AD. Preferably, the decrease is a decrease by at least 10, 20, 30, 40, 50, or 80%. In preferred embodiments, an increase in the level of BRI2 protein as compared to the reference value indicates the risk of developing AD. Preferably, the increase is an increase by at least 10, 20, 30, 40, 50, or 80%.

In preferred embodiments, the level of BRI2 is determined in vitro from a sample from an individual, preferably a biological fluid. Preferably, the sample is blood, urine, saliva, more preferably the sample is cerebral spinal fluid.

The level of BRI2 may be determined using a BRI2 binding compound, such as an antibody. Preferably, the binding compound binds BRI2 at the BRICHOS domain, more preferably at amino acids 140-153 of SEQ ID NO:1. A BRI2 binding compound includes a small molecule, a peptide, a protein, aptamer, an antibody, or an antibody mimic. Antibody mimics refers to molecules capable of mimicking an antibody's ability to bind an antigen, but which are not limited to native antibody structures. Examples of such antibody mimetics include, but are not limited to, Adnectins (i.e., fibronectin based binding molecules), Affibodies, DARPins, Anticalins, Avimers, and Versabodies.

Preferably, the BRI2 binding compound is an antibody. The term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)2, Fv or Fab fragments, or other antigen recognizing immunoglobulin fragments. Preferred antibodies for use in the methods are described further herein.

Binding of BRI2 polypeptide to a BRI2 binding compound is detected by techniques known in the art. For example, in some embodiments, binding is detected using radio-immunoassay, ELISA (enzyme-linked immunosorbant assay), “sandwich” immunoassay, immunoradiometric assay, gel diffusion precipitation reaction, immunodiffusion assay, precipitation reaction, agglutination assay (e.g., gel agglutination assay, hemagglutination assay, etc.), complement fixation assay, immunofluorescence assay, protein A assay, and immunoelectrophoresis assay, or multiplex bead assay (e.g., using Luminex or fluorescent microbeads) or multiplex planar assay (e.g., Mesoscale Discovery).

Preferably, the assay used is a sandwich ELISA. In this assay, the antibody is bound to the solid phase or support, which is then contacted with the sample being tested to extract the antigen from the sample by formation of a binary solid phase antibody:antigen complex. After a suitable incubation period, the solid support is washed to remove the residue of the fluid sample and then contacted with a solution containing a labelled antibody.

Preferably, the assay used is a sandwich bead assay. In this assay, the antibody is bound to beads, which are then contacted with the sample being tested to extract the antigen from the sample by formation of a binary solid phase antibody:antigen complex. After a suitable incubation period, beads are washed to remove the residue of the fluid sample. In some embodiments, the beads are then contacted with a solution containing a known quantity of labelled antibody. Alternatively, the sample itself can be labelled, in which case the binding is detected by the presence of the labelled sample.

Preferably, the BRI2 binding compound of the methods, described herein, is an anti-BRI2 antibody that binds to amino acids 140-153 of SEQ ID NO:1. Accordingly, the disclosure provides anti-BRI2 antibodies that bind to amino acids 140-153 of human BRI2 (SEQ ID NO:1). Amino acids 140-153 of human BRI2 are, thus, the “epitope” or “antigenic determinant” for the antibodies. The term “anti-BRI2 antibody” refers to an antibody, as defined herein, capable of binding to BRI2, more specifically to amino acids 140-153. The term “off-rate” or “K_{d}” refers to the equilibrium dissociation constant of a particular antibody-antigen interaction and is used to describe the binding affinity between a ligand (such as an antibody) and a protein (such as BRI2). The smaller the equilibrium dissociation constant, the more tightly bound the ligand is, or the higher the affinity between ligand and protein. A K_{d} can be measured by surface plasmon resonance, for example, using the BIACORE 1 or the Octet system. Anti-BRI2 antibodies that bind to amino acids 140-153 of human BRI2 have a lower K_{d} for the interaction between the antibody and amino acids 140-153 of BRI2 that for the interaction between the antibody and a different region of BRI2 or to a non-relevant protein. Exemplary anti-BRI2 antibodies and their preparation are described in the examples.

The term “antibody” includes, for example, both naturally occurring and non-naturally occurring antibodies, polyclonal and monoclonal antibodies, chimeric antibodies and wholly synthetic antibodies. Antigen binding fragments of antibodies are also encompassed in the disclosure. The term “antigen-binding fragment” refers to one or more portions of a full-length antibody that retain the ability to bind to the same antigen (i.e., human BRI2) that the antibody binds to, for example, the Fab′, F(ab′)2, Fv or Fab fragments.

Antibodies that bind a particular epitope can be generated by methods known in the art. For example, polyclonal antibodies can be made by the conventional method of immunizing a mammal (e.g., rabbits, mice, rats, sheep, goats). Polyclonal antibodies are then contained in the sera of the immunized animals and can be isolated using standard procedures (e.g., affinity chromatography, immunoprecipitation, size exclusion chromatography, and ion exchange chromatography). Monoclonal antibodies can be made by the conventional method of immunization of a mammal, followed by isolation of plasma B cells producing the monoclonal antibodies of interest and fusion with a myeloma cell (see, e.g., Mishell, B. B., et al. Selected Methods In Cellular Immunology, (W.H. Freeman, ed.) San Francisco (1980)). A peptide having amino acids 140-153 of human BRI2 may be used for immunization in order to produce antibodies which recognize the particular epitope. Screening for recognition of the epitope can be performed using standard immunoassay methods including ELISA techniques, radioimmunoassays, immunofluorescence, immunohistochemistry, and Western blotting. See, Short Protocols in Molecular Biology, Chapter 11, Green Publishing Associates and John Wiley & Sons, Edited by Ausubel, F. M et al. 1992. Alternatively, animals may be immunized with polypeptides which comprise amino acids 140-153 of human BRI2 followed by the screening and/or isolation of antibodies which specifically recognize the particular epitope. In vitro methods of antibody selection, such as antibody phage display, may also be used to generate antibodies recognizing amino acids 140-153 of BRI2 (see, e.g., Schirrmann et al. Molecules 2011 16:412-426).

In preferred embodiments, the antibody is a monoclonal antibody. In preferred embodiments, the antibody is a polyclonal antibody, which does not bind to SEQ ID NO:1 at amino acids outside of 140-153. Such a polyclonal antibody may be produced, e.g., by immunizing an animal with a peptide corresponding to amino acids 140-153 of SEQ ID NO:1 or by affinity purifying the sera from an animal immunized with a BRI2 polypeptide using a peptide corresponding to amino acids 140-153 of SEQ ID NO:1.

Nucleic acid molecules encoding the light chain and heavy chain variable domains of the anti-BRI2 antibodies, described herein, are also encompassed by the disclosure. The nucleic acid molecule encoding the heavy variable region may be fused together with a nucleic acid molecule encoding a constant region of a heavy chain. Similarly, a nucleic acid molecule encoding the light variable region of the antibody may be fused to a nucleic acid molecule encoding a constant region of a light chain. Nucleic acid molecules encoding full-length heavy and/or light chains may then be expressed from a cell into which they have been introduced and the antibody isolated. The nucleic acid molecules may also be used to produce other binding molecules provided by the disclosure, such as chimeric antibodies, single chain antibodies, and antibody binding fragments.

Preferably, the nucleic acid is isolated nucleic acid. The term “isolated nucleic acid” refers to a nucleic acid molecule of genomic, cDNA, or synthetic origin, or a combination thereof, which is separated from other nucleic acid molecules present in the natural source of the nucleic acid. Preferably, an “isolated” nucleic acid is free of sequences located at the 5′ and 3′ ends of the nucleic acid of interest in the genomic DNA of the organism from which the nucleic acid is derived.

A further aspect of the disclosure provides a vector, which comprises a nucleic acid molecule described herein above. The nucleic acid molecule may encode a portion of a light chain or heavy chain (such as a CDR or a variable region), a full-length light or heavy chain, polypeptide that comprises a portion or full-length of a heavy or light chain, or an amino acid sequence of an antibody derivative or antigen-binding fragment. The DNA encoding the amino acid sequence of an antibody chain may be cloned into the vector such that the signal peptide is linked in-frame to the amino terminus of the amino acid sequence of the antibody chain. The signal peptide can be an immunoglobulin signal peptide or a heterologous signal peptide (i.e., a signal peptide from a non-immunoglobulin protein).

The design of the expression vector, including the selection of regulatory sequences, may depend on such factors as the choice of the host cell to be transformed, the level of expression of protein desired, and so forth. Regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. The host cell may be a mammalian, insect, plant, bacterial, or yeast cell.

In preferred embodiments of the methods described herein, the level of BRI2 is determined in vivo, preferably using non-invasive detection. Preferably, the level of BRI2 in the hippocampus is determined. In preferred embodiments, positron emission tomography is used to determine the level of BRI2. Accordingly, a method is provided comprising providing an individual with a PET compatible tracer, wherein the tracer binds BRI2, and scanning the individual with a PET scanner.

PET is a well-known technique to determine the distribution of a tracer in vivo. A radioactive tracer is administered to an individual. The individual is then subjected to a scanning procedure using a PET or PET/CT scanner. Quantification of radiopharmaceutical (radio-tracer) uptake by the target tissue can be performed using methods known in the art (see, e.g., Boellaard R. et al. Journal of Nuclear Medicine, Vol. 45, No. 9, pp 1519-1527, 2004 and U.S. Publications 20100196274 and 20110148861, which are hereby incorporated by reference). The distribution of BRI2 binding tracer can be determined in “normal” individuals to determine a baseline which can be compared to the level in subject suspected of cognitive/memory impairment.

A suitable tracer binds to BRI2 and is preferably a peptide sequence. The tracer is labelled with a short-lived radioactive tracer isotope, such as carbon-11, nitrogen-13, oxygen-15, or fluorine-18. Preferably, the tracer binds to the BRI2 BRICHOS domain, more preferably to amino acids 140-153 of BRI2.

A tracer intensity, which is significantly different from a reference value, indicates a risk of developing Alzheimer's disease. In preferred embodiments, a decrease in tracer intensity (i.e., the level of BRI2 protein as compared to the reference value) indicates the risk of developing AD. Preferably, the decrease is a decrease by at least 10, 20, 30, 40, 50, or 80%. In preferred embodiments, an increase in tracer intensity indicates the risk of developing AD. Preferably, the increase is an increase by at least 10, 20, 30, 40, 50, or 80%. Methods for establishing a diagnostic based on PET analysis are known in the art (see, e.g., US 20100196274 and US 20100249418).

In preferred embodiments, methods are provided for treating an individual comprising a) determining the level of BRI2 polypeptide in the individual, as described herein, and b) treating an individual having an altered BRI2 polypeptide level as compared to a reference sample with an Alzheimer's disease treatment. Preferably, individuals having a reduced BRI2 polypeptide level as compared to a reference sample are treated for Alzheimer's. Preferably, individuals having an increased BRI2 polypeptide level as compared to a reference sample are treated for Alzheimer's. The treatments include administration of a therapeutically effective amount of an acetyl cholinesterase inhibitor, solanezumab, memantine, or an NMDA receptor antagonist. Preferably, the treatment is selected from donepezil (brand name ARICEPT™), galantamine (RAZADYNE™), and rivastigmine (EXELON™). The administration of such compounds is described in U.S. Publication Nos. 20060160079 and 20120323214, which are hereby incorporated by reference. Preventive treatments can include functional foods such as those described in Scheltens J Alzheimers Dis. 2012; 31(1):225-36. Other preferred treatments comprise compounds which reduce BRI2 protein levels, in particular the amount or activity of nonfunctional BRI2, as described herein.

In a further embodiment of the disclosure, methods are provided for treating Alzheimer's disease in an individual, comprising administering to an individual in need thereof a therapeutically effective amount of a compound, which reduces the level of BRI2 protein, in particular the BRI2 polypeptide containing the BRICHOS domain. Preferably, the compound reduces the amount of nonfunctional or abnormal BRI2. As already described herein, non-functional or abnormal BRI2 may acquire a new function, such as protein binding, dimerisation or aggregation, which thereby can reduce its binding capacity to proteins such as amyloid precursor protein (APP), which would have inhibited the cleavage of APP into abeta-species. Preferably, the compound reduces this new function or activity of abnormal BRI2. If, for example, abnormally high levels of BRI2 result in BRI2 accumulation, the compound may reduce BRI2 levels, which results in a reduction in BRI2 accumulation, i.e., “non-functional” BRI2.

The compounds include polypeptides, small molecules, and nucleic acid based inhibitors. In some embodiments, the compound is a deglycosylation agent. Preferably, the compound is a nucleic acid molecule (such as an antisense oligonucleotide, an RNA interference molecule) or a binding molecule (e.g., an antibody or antibody fragment), kinase or peptide inhibitors with activity for sulfotransferases, or sulfamates, heparin mimetics or other substrate analogue mimetics (examples of compounds may be found in Muthana et al. ACS Chem. Biol. 2012 Jan. 20; 7(1):31-43).

In some embodiments, the compound is a nucleic acid molecule whose presence in a cell causes the degradation of or inhibits the function, transcription, or translation of its target gene, i.e., BRI2, in a sequence-specific manner. Exemplary nucleic acid molecules include aptamers, siRNA, artificial microRNA, interfering RNA or RNAi, dsRNA, ribozymes, antisense oligonucleotides, and DNA expression cassettes encoding the nucleic acid molecules.

Preferably, the nucleic acid molecule is an antisense oligonucleotide. Antisense oligonucleotides (AONs) generally inhibit their target by binding target mRNA and sterically blocking expression by obstructing the ribosome. AONs can also inhibit their target by binding target mRNA, thus, forming a DNA-RNA hybrid that can be a substance for RNase H. AONs may also be produced as composite structures of two or more oligonucleotides, modified oligonucleotides, oligonucleotides, oligonucleotide mimetics, or regions or portions thereof. Such compounds have also been referred to in the art as hybrids or gapmers. Methods for designing and modifying such gapmers are described in, for example, U.S. Patent Publication Nos. 20110092572 and 20100234451.

AONs typically comprise between 12 to 80, preferably between 15 to 40 nucleobases. Preferably, the AONs comprise a stretch of at least 8 nucleobases having 100% complementarity with the target mRNA.

Preferably, the nucleic acid molecule is an RNAi molecule, i.e., RNA interference molecule. Preferred RNAi molecules include siRNA, shRNA, and artificial miRNA.

siRNA comprises a double stranded structure typically containing 15 to 50 base pairs and preferably 19 to 25 base pairs and having a nucleotide sequence identical or nearly identical to an expressed target gene or RNA within the cell. An siRNA may be composed of two annealed polynucleotides or a single polynucleotide that forms a hairpin structure. As used herein, “shRNA” or “small hairpin RNA” (also called stem loop) is a type of siRNA. In one embodiment, these shRNAs are composed of a short, e.g., about 10 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The design and production of siRNA molecules is well known to one of skill in the art (Hajeri P B, Singh S K. Drug Discov Today. 2009 14(17-18):851-8). Methods of administration of therapeutic siRNA is also well known to one of skill in the art (Manjunath N, and Dykxhoorn D M. Discov Med. 2010 May; 9(48):418-30; Guo J et al. Mol. Biosyst. 2010 Jul. 15; 6(7):1143-61). siRNA molecule comprises an antisense strand having about 15 to about 30 (e.g., about 15, 16, 17, 18, 19, 20, 21, 22, 23, 24, 25, 26, 27, 28, 29, or 30) nucleotides, wherein the antisense strand is complementary to a RNA sequence or a portion thereof encoding.

The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle. Liposomes are well known in the art and many variants are available for gene transfer purposes.

Vectors comprising the nucleic acids are also provided. A “vector” is a recombinant nucleic acid construct, such as plasmid, phase genome, virus genome, cosmid, or artificial chromosome, to which another DNA segment may be attached. The ten “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus (AAV), pox, baculovirus, vaccinia, herpes simplex, Epstein-Barr and adenovirus vectors. Vector sequences may also contain one or more regulatory regions, and/or selectable markers useful in selecting, measuring, and monitoring nucleic acid transfer results (transfer to which tissues, duration of expression, etc.). Lentiviruses have been previously described for transgene delivery to the hippocampus (van Hooijdonk BMC Neuroscience 2009, 10:2)

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al. Trends in Biotechnology 11:205-210 (1993)).

In a preferred embodiment, a compound is provided directly to the hippocampus. The compound may be delivered by way of a catheter or other delivery device having one end implanted in a tissue, e.g., the brain by, for example, intracranial infusion. Such methods are known in the art and are further described in U.S. Publications 20120116360 and 20120209110, which are hereby incorporated by reference.

A compound, as described herein, may also be administered into the cerebral spinal fluid. Such compounds are preferably linked to molecules that preferentially bind hippocampal cells (e.g., molecules that bind hippocampal specific cell surface molecules).

Methods that use a catheter to deliver a therapeutic agent to the brain generally involve inserting the catheter into the brain and delivering the composition to the desired location. To accurately place the catheter and avoid unintended injury to the brain, surgeons typically use stereotactic apparatus/procedures (see, e.g., U.S. Pat. No. 4,350,159). During a typical implantation procedure, an incision may be made in the scalp to expose the patient's skull. After forming a burr hole through the skull, the catheter may be inserted into the brain.

Actual dosage levels of the pharmaceutical preparations, described herein, may be varied so as to obtain an amount of the active ingredient, which is effective to achieve the desired therapeutic response for a particular patient, composition, and mode of administration, without being toxic to the patient. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds, described herein, at levels lower than that required in order to achieve the desired therapeutic effect and gradually increase the dosage until the desired effect is achieved.

The effect of the disclosed treatments on Alzheimer's disease (AD) in humans can be examined, for example, through the use of a cognitive outcome measure in conjunction with a global assessment (see, e.g., Leber P: GUIDELINES FOR THE CLINICAL EVALUATION OF ANTIDEMENTIA DRUGS, 1st draft, Rockville, Md., US Food and Drug Administration, 1990). A number of specific established tests that can be used alone or in combination to evaluate a patient's responsiveness to an agent are known in the art (see, e.g., Van Dyke et al. Am. J. Geriatr. Psychiatry 14:5 (2006). For example, responsiveness to an agent can be evaluated using the Severe Impairment Battery (SIB), a test used to measure cognitive change in patients with more severe AD (see, e.g., Schmitt et al. Alzheimer Dis. Assoc. Disord 1997; 11(suppl 2):51-56). Responsiveness to an agent can also be measured using the 19-item Alzheimer's Disease Cooperative Study-Activities of Daily Living inventory (ADCSADL19), a 19-item inventory that measures the level of independence in performing activities of daily living, designed and validated for later stages of dementia (see, e.g., Galasko et al. J. Int. Neuropsychol Soc. 2005; 11:446-453). Responsiveness to therapy can also be measured using the Clinician's Interview-Based Impression of Change Plus Caregiver Input (CIBIC-Plus), a seven-point global change rating based on structured interviews with both patient and caregiver (see, e.g., Schneider et al. Alzheimer Dis. Assoc. Disord 1997; 11(suppl 2):22-32). Response on cognition in early AD stages can also be assessed using memory function domain score (z-score) based on the neurpsychological test battery. This domain includes Rey Auditory Verbal Learning Test immediate recall, delayed recall and recognition performance, and Wechsler Memory Scale-revised (WMS-r) verbal paired associates immediate and delayed recall. Other outcome measures can include executive function domain score (z-score) based on the WMS-r Digit Span, Trail Making Tests parts A and B (DELIS KAPLAN EXECUTIVE FUNCTION SYSTEM™ condition 2 and condition 4, respectively), Category Fluency, the Controlled OralWord Association Test, orientation task of the ADAS-cog and the Letter Digit Substitution Test. Cognition is preferably evaluated by a computerised test (e.g., Applicability of the CANTAB-PAL computerized memory test in identifying amnestic mild cognitive impairment and Alzheimer's disease. Junkkila J, Oja S, Laine M, Karrasch M. Dement Geriatr Cogn Disord. 2012; 34(2):83-9). In addition, electrical brain activity can be measured directly at the skull with electroencephalography (EEG) as a measure of synaptic connectivity (Stam C J (2010) Int J Psychophys 77, 186-194).

In a further aspect, the disclosure provides methods for identification of compounds for the treatment of an Alzheimer's disease, the method comprising: (a) administering one or more candidate compounds to an animal model of Alzheimer's disease and (b) assessing changes in BRI2 in the animal model relative to measures of BRI2 in a control animal. Preferably, a candidate compound is selected that induces a change in BRI2 toward measures of BRI2 in a control animal. Preferably, the treatment is for during preclinical stages of AD.

Any animal model of Alzheimer's disease may be used in the described method (see, e.g., Gotz et al. J Mol. Psychiatry. 2004 July; 9(7):664-83; Richardson and Burns, ILAR J. 2002; 43(2):89-99). Preferred animal models and methods of screening are described in U.S. Pat. No. 5,720,936, which is hereby incorporated by reference. Preferably, the animal model is a transgenic mouse having integrated into the chromosome a nucleic acid construct. Preferably, the animal models overexpress APP, mutated APP, Tau or Presinilin genes, alone or combined, such as CRND8 mice, B6C3-Tg, Tg2576 mice, or the triple transgenic model of AD (3×Tg-AD)). Alternative models are BRI2 knock-in models (Tamayev R et al. J. Neurosci. 2010 Nov. 3; 30(44):14915-24).

Candidate compounds are administered by any means to the animals, for example, by injection or in the drinking water. The changes in BRI2 levels may be assessed by any means, such as the in vitro and in vivo methods described herein. Alternatively, the animals may be sacrificed and the levels of BRI2 may be determined on tissue samples, preferably from the hippocampus, e.g., by Elisa or western blots.

Preferably, the compounds increase BRI2 protein levels. Preferably, the compounds decrease BRI2 protein levels resulting in a decrease in nonfunctional or abnormal BRI2. Preferably, the compounds reduce BRI2 aggregation and/or reduce BRI2 glycosylation.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound, as defined herein, may comprise additional component(s) than the ones specifically identified, the additional component(s) not altering the unique characteristic of the disclosure.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

All patent and literature references cited in the present specification are hereby incorporated by reference in their entirety.

The disclosure is further explained in the following examples. These examples do not limit the scope of the disclosure, but merely serve to clarify the disclosure.

### EXAMPLES

### Example 1

### Label-Free GeLC-MS/MS-Based Proteomics Analysis

In order to identify novel relevant pathways involved in AD we analyzed by hypothesis-free proteomics approach CSF from patients with SMC, MCI-S, MCI-AD and AD. The spectral count of BRI2 peptides in CSF was significantly increased in MCI-AD and AD patients compared to SMC and MCI-S patients (FIG. 1B).

The proteomic CSF data similarly showed an increase in BRI2 peptides spanning aa 110-221 in MCI-AD and AD compared to SMC and MCI-S patients suggesting that the BRI2 changes found in the brain might be reflected in CSF.

### Example 2

### Antibody Characterization

Analysis of BRI2₁₄₀₋₁₅₃ peptide using Basic Local Alignment Search Tool (BLAST, NCBI) showed an e-value of 3e-08 for BRI2. The e-values of other proteins were higher than 0.01 indicating that BRI2₁₄₀₋₁₅₃ is a unique sequence for human BRI2. Polyclonal antibodies raised against BRI2₁₄₀₋₁₅₃ detected bands of 40, 45 and 52 kDa in AD and controls human brain homogenates on Western Blots (FIGS. 1C-D). Additional bands were detected at 15, 25, 75 kDa using Protein A-purified BRI2₁₄₀₋₁₅₃ (FIG. 1D) and the reactivity of all bands was dramatically decreased after peptide pre-absorption of the antibody (FIG. 1E). The same 40, 45 and 52 kDa bands were observed when a monoclonal antibody raised against BRI2₁₁₁₋₁₅₃ was used (FIG. 1F). Reactivity against recombinant human BRI₇₆₋₂₆₆ protein showed reactive bands at 10, 15, 20, 30, 40 and 90 kDa using polyclonal antibodies against BRI2₁₄₀₋₁₅₃ (FIG. 1G) or BRI2₁₁₃₋₂₃₁ (FIG. 9B). Specificity of all other anti-BRI2 antibodies towards the 45 kDa band was further confirmed by a dramatic decrease in immunoreactivity after pre-absorption with recombinant human BRI2₁₄₀₋₁₅₃ or BRI2₇₆₋₂₂₆ (FIG. 9A).

Immunohistochemical examination of AD and control brain tissue for the presence and localization of BRI2, showed similar BRI2 deposition in plaques using polyclonal protein A-purified anti-BRI2₁₄₀₋₁₅₃, monoclonal protein G-purified anti-BRI2₁₄₀₋₁₅₃, monoclonal IgM-purified anti-BRI2₁₁₁₋₁₅₃ or polyclonal protein G-purified BRI2₁₁₃₋₂₃₁ antibodies (Supplementary FIG. 1C). Reactivity of the polyclonal protein A-purified anti-BRI2₁₄₀₋₁₅₃ antibody to amyloid plaques was abolished after peptide pre-absorption of the antibody (FIG. 9D).

Antibody characterization showed specific BRI2 bands at 40, 45 and 52 kDa in hippocampus homogenates for all the antibodies tested, which is in line with previous studies (Kim et al. 1999; Martin et al. 2008, 2009; Tsachaki et al. 2010). However, based on its aa sequence, the expected molecular weight of BRI2 is 30 kDa. A recent transgenic cell culture study proposed that glycosylation of BRI2 at aa Asn170 might explain the higher molecular weights observed in the previous studies. However, deglycosylation of BRI2 decreases its molecular weight by only 2 kDa (Tsachaki et al. 2011) suggesting the involvement of other mechanisms in the generation of higher molecular weight forms of BRI2. Intriguingly, Western blot analysis of the recombinant BRI2 ectodomain under denatured conditions showed also bands of higher molecular weight, which suggests that BRI2 is able to aggregate. We propose that BRI2 may undergo additional post-translational modifications or can constitute aggregates with itself and/or other proteins accounting for the relatively higher molecular bands of 45 and 52 kDa.

### Example 3

### BRI2 Accumulates in AD Hippocampus in Early Pathological Stages and Associates with Amyloid Plaques

In order to analyze the location of the increase in BRI2 reactivity in AD patients we performed BRI2 immunostainings on post-mortem hippocampus brain sections of 34 patients (30 of them were the same patients as analyzed by Western blot). Three different areas within the hippocampus (CA4 to 2, CA1 and Subiculum) were examined (FIG. 3A). BRI2 staining in control patients was mainly observed in neuronal cytosol or in astrocytes. Microscopical observations showed BRI2 deposition in the three hippocampal areas in AD patients, but not in control patients (FIGS. 3B and 3C). This deposition appeared extracellular and had a rounded shape. We next asked whether extracellular BRI2 deposition was related to Aβ deposition. To this end, we performed double immunohistochemical stainings detecting BRI2 as well as the Aβ peptide. The results showed a clear association between BRI2 and Aβ immunoreactivity (FIG. 3C, third row). Detailed analysis of this association revealed that approximately 50% of the Aβ plaques were BRI2 positive (FIG. 11).

Quantification of BRI2 deposition revealed a significant increase in AD compared to control patients, when patients were grouped either to the pathological (FIG. 4) or clinical (FIG. 12) diagnosis.

There was a significant correlation between the extent of BRI2 staining and the levels of the 45 kDa BRI2 observed by Western blot in all hippocampus areas (FIG. 10). We next questioned if BRI2 deposition starts in early stages of the AD pathology. The data in FIG. 5 show that BRI2 deposition in the hippocampus was present already in the middle stages (III/IV-3). The maximal BRI2 values are found at Braak stage 1V (FIG. 5A). BRI2 deposition also increased with amyloid progression (Amyloid staging according to Thal (Thal et al. 2006)), i.e., the highest amount of BRI2 deposition was observed in the most advanced stage of the disease (FIG. 5B). Collectively, these results revealed that in AD hippocampus there is a deposition of BRI2 protein associated with amyloid plaques, which is not present in control tissue. The data additionally showed that BRI2 deposition starts already in early stages of the AD pathology. Extracellular BRI2 immunoreactivity was not present in brain tissue from patients with frontotemporal dementia, Parkinson's disease, Pick's disease and Gerstmann-Sträussler-Scheinker syndrome (GSS).

Our Western blot results showed specific increased levels of the 45 kDa BRI2 band in AD hippocampus compared to controls based on both clinical and pathological diagnosis of AD. Immunohistochemical analysis confirmed those results, as we observed BRI2 deposition in the hippocampus of AD patients, but not in control cases. These results are in agreement with the BRI2 staining previously observed in the temporal cortex of one Alzheimer's disease case (Akiyama et al. 2004). A previous small-scale (n=4) immunohistochemical study compared the presence of BRI2 in AD hippocampus and control cases but no difference was found (Lashley et al. 2008). The discrepancy found could be explain by the antibody used in the latter study, which was raised against the C-terminal part of the BRI2 BRICHOS domain (BRI2223-233). BRI2223 is involved in the formation of disulfide bonds and loop like structures (Garringer et al. 2010) that could mask the epitope recognized by that antibody when the protein is aggregated. Moreover, the results could be specific for our antibodies, which were generated against a BRI2 specific sequence in the center of the BRICHOS domain. There was a significant correlation between increased levels of the BRI2-positive bands in Western blot and the immunohistochemistry results, which suggests that BRI2 immunostaining represented deposition of the 45 kDa form of BRI2.

### Example 4

### Levels of BRI2 Processing Enzymes Furin, ADAM10 and SPPL2b are Also Changed in AD Human Hippocampus

BRI2 undergoes three consecutive cleavages performed by a furin-like protease, ADAM10 and SPPL2b leading to the secretion of different molecular weight peptides (12, 13) (FIG. 1A). The higher intensity of the 50 kDa-lower band on Western blot suggested higher levels of un-processed BRI2 in AD patients (FIG. 2A). Thus, we wondered if the levels of furin, ADAM10 and SPPL2b were also different between AD and control patients. To this end, we analyzed the levels of these proteins by Western blot in the same hippocampus homogenates as tested for BRI2 reactivity and correlated to BRI2 expression (FIG. 6A). Furin levels were decreased in AD patients although this difference was not significant (FIG. 6D). ADAM10 was significantly decreased in AD compared to non-AD patients (FIG. 6B). SPPL2b was strongly increased in AD hippocampus homogenates (FIG. 6C). Interestingly, the data revealed a correlation between the levels of ADAM10 with the levels of both furin (FIG. 6E) and BRI2 (FIG. 6F). No significant correlation was found between the levels of furin and BRI2 (FIG. 6G). The increase in SPPL2b did not correlate with any of the other proteins analyzed (FIG. 14).

### Example 5

### BRI2 Binding to APP is Absent in AD Hippocampus

Since BRI2 is able to bind APP in vitro/mice models we wanted to analyze if this binding also occurs in human brains and if it is conserved in AD cases. Immunopurification of BRI2 from human hippocampus revealed that BRI2 bound APP in the control cases. Interestingly, BRI2-APP complexes were not present in the hippocampus from AD cases (FIG. 7A). Similar results were obtained using a different immunopurification procedure with other BRI2 antibody in two other AD and two other non-AD patients (FIG. 7B).

### Example 6

### BRI2 Reduction in CSF of AD Patients

Next, we set out to analyse BRI with a single binder beads assay. In this method, protein targets are detected in a suspension beads assay by biotinylating all proteins present in the sample. The target proteins are bound by individual specific antibodies coated to beads, and the biotin present on the bound proteins is detected by fluorescently labelled streptavidin. For this experiment, 48 CSF samples with different biochemical Alzheimer profiles were biotinylated. “Control” had normal CSF Aβ42 and Tau levels, which are abnormal (decreased and increased respectively) in “AD-profile.” Fluorescence signal obtained by anti-BRI2₇₆₋₂₂₆ coupled beads was correlated to levels of Aβ42, Tau and phosphorylated Tau. The results showed that the BRI2 positive signal was decreased in patients with an AD biomarker profile compared to patients with a control profile, and that this signal in CSF correlated positively to levels of Aβ42 (r=0.33, P<0.05), but not to Tau and phosphorylated Tau (data not shown).

Materials and Methods

Post-Mortem Brain Tissue

Post-mortem brain material was obtained from the Netherlands Brain Bank (Amsterdam, The Netherlands). All donors (n=40) or their next of kin provided written informed consent for brain autopsy and use of tissue and medical records for research purposes. Clinical diagnosis was defined according to DSM-III-R criteria and the severity of dementia prior to death had been evaluated with the Global Deterioration Scale of Reisberg (Reisberg et al. 1982). Neuropathological evaluation was performed on formalin-fixed, paraffin-embedded tissue from different brain areas. The distribution and the density of neurofibrillary tangles (NFTs) were determined using Bodian staining and immunohistochemistry for hyperphosphorylated tau. Senile plaques were stained with the methenamine silver method (Yamaguchi et al. 1990). Staging of AD was evaluated according to the Braak criteria for NFTs (Braak & Braak, 1991; Braak et al. 2006) and according to Thal criteria for amyloid deposition (Thal et al. 2006). Clinical and pathological diagnosis, gender, age, post-mortem interval, Braak and Thal scores for NFTs and amyloid load of all cases are listed in Table I. Both clinical and pathological diagnoses were used as outcome data to analyze the results.

Frozen brain hippocampus tissue blocks were present from 31 of these cases. The tissue was homogenized with Mammalian Protein Extraction Reagent (M-PER, 0.1 g/ml, Thermo Scientific, Waltham, USA) containing EDTA-free Protease Inhibitor Cocktail (1:25, Roche, Basel, Germany). Human brain homogenates (HBH) were centrifuged at 10,500 g for 30 min at 4° C. The protein content in the supernatant was quantified using bovine serum albumin (BSA) standards (Thermo Scientific, Waltham, USA) and the Bio-Rad Protein Assay (Bio-Rad, Hercules, USA). Samples were stored at −80° C. until further analysis.

CSF Samples

CSF material (n=18) was obtained from NUBIN (NeuroUnit Biomarkers for Inflammation and Neurodegeneration) VUmc biobank (Amsterdam, The Netherlands). Clinical assessment of subjects was done as previously described (Mulder et al. 2010). CSF samples were stored in agreement with BioMS-eu guidelines (Teunissen et al. 2009, 2011; Del Campo et al. 2012). AD patients (n=5) and non-demented subjects with subjective memory complaints (SMC; n=4) were selected. Patients with mild cognitive impairment (MCI) who after two year follow up converted to AD (MCI-AD; n=5) or remained stable (MCI-S; n=4) were also included. Age, gender, biomarker levels as well as Mini Mental State Examination (MMSE) score at baseline and follow up of all cases used are listed in Table II. The ethical review board of the VUmc approved the study and all subjects gave written informed consent.

Mass Spectrometry Analysis of CSF

CSF samples were analyzed by label-free GeLC-MS/MS-based proteomics and normalized spectral counting as previously described (Fratantoni et al. 2010). The data obtained were processed and analyzed as described before (Pham et al. 2010). The global protein profiling results of the CSF proteomics screen will be reported elsewhere (Chiasserini et al. in preparation). BRI2 was one of the proteins identified.

Antibody Characterization and Purification.

Polyclonal and monoclonal antibodies against human BRI2 were raised by immunizing rabbits and mice with Limulus Polyphemus Hemocyanin (LPH)-conjugated synthetic peptides corresponding to the human BRI amino acids 140-153 (BioGenes GmbH, Berlin, Germany) which resides in the BRI2 BRICHOS domain (FIG. 1A). Antibodies were purified using HiTrap™ Protein A or Protein G HP Columns (GE Healthcare, Amersham, UK) on GE Pharmacia ÄKTA™ Purifier (GE Healthcare, Amersham, UK). Affinity purification of polyclonal antibody BRI2140-153 was performed using antigen-peptide-conjugated sepharose columns (BioGenes GmbH, Berlin, Germany). A monoclonal antibody (anti-BRI2111-153, IgM) was produced by immunizing mice with recombinant human BRI protein (76-266) expressed and purified from E. coli as described (Korth et al. 1997). Monoclonal antibody was purified using HiTrap™ IgM HP Columns (GE Healthcare, Amersham, UK). Goat polyclonal antibody against BRI2113-231 was a generous gift from Dr. Janne Johansson (Karolinska instituted, Stockholm, Sweden).

Antibody specificity on Western blot of the five different purified antibodies (Polyclonal protein A-purified anti-BRI2140-153, Polyclonal affinity-purified anti-BRI2140-153, Monoclonal Protein G-purified anti-BRI2140-153, Polyclonal protein G-purified anti-BRI2113-231 and Monoclonal IgM-purified anti-BRI2111-153) was analyzed through reactivity comparison towards BRI2 in HBH and recombinant BRI76-266. Specificity was further tested by comparison with antibodies that were pre-absorbed with recombinant human BRI2140-153 or BRI276-226 (1:10 w/w, 8 hours).

Western Blotting.

HBH (12 μg) samples or BRI76-266 recombinant protein (2 μg) were prepared in sample buffer (2% SDS, 0.03 M Tris, 5% 2-Mercaptoethanol, 10% glycerol, bromophenol blue) and heated 5 min at 95° C. Electrophoresis of HBH was carried out using pre-cast NuPAGE Bis-Tris Mini Gels 4-12% (1 mm, 4-12%; Invitrogen, Carlsbad, USA) or 10% SDS-PAGE mini gels. Next, proteins were transferred to polyvinylidene fluoride membranes (PVDF; Millipore, Bedford, USA) that were subsequently blocked for 30 minutes with blocking buffer (5% (w/v) non-fat dried milk in PBS-Tween 0.5% (v/v) (PBS-T)), and incubated with either affinity-purified polyclonal rabbit anti-BRI2140-153 (2.3 μg/ml), monoclonal mouse anti-Furin B6 (1:1000, Santa Cruz Biotechnology, Santa Cruz, USA), polyclonal rabbit anti-ADAM10 ab1997 (1:2000, Abcam, Cambridge, UK), polyclonal rabbit anti-SPPL2b (1:1000, Aviva system biology, San Diego, USA), monoclonal mouse anti-APP clone 22C11 (1:10000, clone 22C11, Millipore, Bedford, USA) or monoclonal mouse anti-Actin (clone AC-40, 1:1000, Sigma-Aldrich, Saint Louis, USA) in blocking buffer overnight. After washing with washing buffer (0.05% (w/v) Milk in PBS-T), membranes were incubated during 1 hour with polyclonal swine anti-rabbit IgG/HRP (1:3000, DAKO, Glostrup, Denmark) or goat anti-mouse IgG/HRP (1:1000, DAKO, Glostrup, Denmark) in blocking buffer. Protein bands were detected with ECLTM Western Blotting detection kit (GE Healthcare, Amersham, UK). Immunoblot films were scanned and signal quantification was performed using ImageJ 1.45 (NIH, Bethesda, USA). Signal intensity was normalized by the actin signal intensity.

BRI2 Immunoprecipitation

In order to precipitate BRI2 from human hippocampus, individual HBH (10 μg) were pre-cleared with 5 μl of Dynabeads Protein G (Life technologies, Carlsbad, USA) during 1 hour at room temperature. Cleared HBH were incubated with 2 μl of Protein G-purified goat anti-BRI2137-231 or control rabbit polyclonal antibody in TBS buffer (1:9 v/v, 50 mM Tris, 150 mM NaCl, pH 7.6) with EDTA 2 mM and 0.05% Triton 100× overnight at 4° C. Antibody-bound protein complexes were then incubated with 20 μl of Dynabeads Protein G for 1 hour at room temperature and washed 4 times with TBS buffer. Beads were resuspended in 60 μl sample buffer and APP in the precipitates was analyzed by gel-electrophoresis and Western Blotting. Another immunoprecipitation of BRI2 from HBH was performed using Protein G-purified rabbit anti-BRI2140-153 following the manufacturer's instructions of DYNABEADS® Co-Immunoprecipitation Kit (Life technologies, Carlsbad, USA). BRI2 precipitates were analyzed for APP by Western blot, as described above.

Immunohistochemistry

Formalin-fixed and paraffin-embedded hippocampus sections (5 μm) were mounted on Superfrost plus tissue slides (Menzel-Glaser, Braunschweig, Germany) and dried overnight at 37° C. For all stainings, sections were deparaffinized and subsequently immersed in 0.3% H2O2 in methanol for 30 minutes to quench endogenous peroxidase activity. Sections were boiled in a microwave in 10 mmol/L pH 6.0 sodium citrate buffer during 10 minutes for antigen retrieval and incubated 10 minutes with normal swine serum (1:10; DAKO, Glostrup, Denmark). Phosphate-buffered saline (PBS) containing 1% (w/v) bovine serum albumin (Boehringer Mannheim, Germany) was used as diluent for normal swine serum and antibodies. Sections were incubated with protein A-purified rabbit anti-BRI2140-153 (9.2 μg/ml) overnight at 4° C. After washing with PBS, sections were incubated with biotin-conjugated swine anti-rabbit F(ab)₂ (1:300, DAKO, Glostrup, Denmark). Next, sections were incubated with streptavidin-biotin horseradish peroxidase complex (strept ABComplex/HRP, 1:100; DAKO, Glostrup, Denmark) for 60 minutes. Sections were then stabilized for 5 minutes with Tris-HCl buffer (0.2 M pH 8.5). Color was developed using Liquid Permanent Red (LPR, 17 minutes, DAKO, Glostrup, Denmark) as chromogen. Nuclei were stained with hematoxylin and sections were mounted using Aquamount (BDH Laboratories Supplies, Dorset, UK). Antibody specificity was evaluated by comparing immunohistochemistry patterns using all except the polyclonal affinity-purified anti-BRI2140-153. Antibody pre-absorption with the antigenic peptide was also performed for the polyclonal protein A-purified anti-BRI2140-153 antibody. Negative controls for all single and double immunostainings were generated by omission of primary antibodies.

Double Immunohistochemistry: BRI2 with Aβ

To determine co-localization of BRI2 with amyloid plaques, sections were incubated with rabbit protein A-purified anti-BRI2140-153 (9.2 μg/ml) simultaneously with mouse anti-Aβ1-17 (2.57 μg/ml; VUmc; Verwey et al. manuscript in preparation) overnight at 4° C. After washing with PBS, sections were incubated with biotin-conjugated swine anti rabbit-F(ab)₂ (1:300 dilution) together with EnVision solution (goat anti-mouse HRP, undiluted; DAKO, Glostrup, Denmark) during 60 minutes for the detection of primary antibodies. Further processing was performed, as described above. Reactivity against Aβ1-17 was developed using 3,3-diaminobenzidine (DAB, 0.1 mg/ml, 0.02% H2O2, 2 minutes; Sigma, St. Louis, Mo.) as chromogen. Sections were then intensively washed with MilliQ water and Tris-HCl buffer (0.2 M pH 8.5) during 5 minutes, before visualization of BRI2 using liquid permanent red.

Evaluation of Stainings

Quantitative analysis of BRI2 immunoreactive plaques was performed on single stained slides by manually counting the number of BRI2 depositions in different regions of the hippocampus (CA4-CA2, CA1 and Subiculum) and correcting for the size of the area. Double (BRI2 and Aβ) stainings were used to determine the association of BRI2 with Aβ containing plaques. Data were corrected for the size of the area and the percentage of BRI2 positive plaques was calculated for each hippocampus area. Staining and counting was performed twice by two independent researchers, who were unaware of the diagnosis and specifics of the cases.

Statistical Analysis

Statistical analyses were performed on SPSS version 16.0 (Chicago, USA) using non-parametric Student's t-test (two groups analysis) or one-way ANOVA (multiple group analysis) to analyze group differences. Correlation analysis were done using Spearman's test. Values with p<0.05 were considered significant.

### REFERENCES

Akiyama H, Kondo H, Arai T, Ikeda K, Kato M, Iseki E, Schwab C & McGeer P L (2004) Expression of BRI, the normal precursor of the amyloid protein of familial British dementia, in human brain. Acta neuropathologica 107: 53-8.
Bekris L M, Lutz F, Li G, Galasko D R, Farlow M R, Quinn J F, Kaye J A, Leverenz J B, Tsuang D W, Montine T J, Peskind E R & Yu C-E (2012) ADAM10 expression and promoter haplotype in Alzheimer's disease. Neurobiology of aging 33: 2229.e1-2229.e9.
Bernstein H-G, Bukowska A, Krell D, Bogerts B, Ansorge S & Lendeckel U (2003) Comparative localization of ADAMs 10 and 15 in human cerebral cortex normal aging, Alzheimer disease and Down syndrome. Journal of neurocytology 32: 153-60.
Bernstein H-G, Stricker R, Lendeckel U, Bertram I, Dobrowolny H, Steiner J, Bogerts B & Reiser G (2009) Reduced neuronal co-localisation of nardilysin and the putative alpha-secretases ADAM10 and ADAM17 in Alzheimer's disease and Down syndrome brains. Age (Dordrecht, Netherlands) 31: 11-25.
Braak H, Alafuzoff I, Arzberger T, Kretzschmar H & Del Tredici K (2006) Staging of Alzheimer disease-associated neurofibrillary pathology using paraffin sections and immunocytochemistry. Acta neuropathologica 112: 389-404.
Braak H & Braak E (1991) Neuropathological staging of Alzheimer-related changes. Acta neuropathologica 82: 239-59.
Del Campo M, Mollenhauer B, Bertolotto A, Engelborghs S, Hampel H, Simonsen A H, Kapaki E, Kruse N, Le Bastard N, Lehmann S, Molinuevo J L, Parnetti L, Perret-Liaudet A, Sáez-Valero J, Saka E, Urbani A, Vanmechelen E, Verbeek M, Visser P J & Teunissen C (2012) Recommendations to standardize preanalytical confounding factors in Alzheimer's and Parkinson's disease cerebrospinal fluid biomarkers: an update. Biomarkers in medicine 6: 419-30.
Endres K & Fahrenholz F (2012) Regulation of alpha-secretase ADAM10 expression and activity. Experimental brain research. Experimentelle Hirnforschung. Expérimentation cérébrale 217: 343-52.
Fotinopoulou A, Tsachaki M, Vlavaki M, Poulopoulos A, Rostagno A, Frangione B, Ghiso J & Efthimiopoulos S (2005) BRI2 interacts with amyloid precursor protein (APP) and regulates amyloid beta (Abeta) production. The Journal of biological chemistry 280: 30768-72.
Fratantoni S A, Piersma S R & Jimenez C R (2010) Comparison of the performance of two affinity depletion spin filters for quantitative proteomics of CSF: Evaluation of sensitivity and reproducibility of CSF analysis using GeLC-MS/MS and spectral counting. Proteomics. Clinical applications 4: 613-7.
Friedmann E, Hauben E, Maylandt K, Schleeger S, Vreugde S, Lichtenthaler S F, Kuhn P-H, Stauffer D, Rovelli G & Martoglio B (2006) SPPL2a and SPPL2b promote intramembrane proteolysis of TNFalpha in activated dendritic cells to trigger IL-12 production. Nature cell biology 8: 843-8.
Garringer H J, Murrell J, D'Adamio L, Ghetti B & Vidal R (2010) Modeling familial British and Danish dementia. Brain structure & function 214: 235-44.
Hwang E M, Kim S-K, Sohn J-H, Lee J Y, Kim Y, Kim Y S & Mook-Jung I (2006) Furin is an endogenous regulator of alpha-secretase associated APP processing. Biochemical and biophysical research communications 349: 654-9.
Jahn H, Wittke S, Zürbig P, Raedler T J, Arlt S, Kellmann M, Mullen W, Eichenlaub M, Mischak H & Wiedemann K (2011) Peptide Fingerprinting of Alzheimer's Disease in Cerebrospinal Fluid Identification and Prospective Evaluation of New Synaptic Biomarkers. PLoS ONE 6: e26540.
Kilger E, Buehler A, Woelfing H, Kumar S, Kaeser S A, Nagarathinam A, Walter J, Jucker M & Coomaraswamy J (2011) BRI2 regulates {beta}-amyloid degradation by increasing levels of secreted insulin degrading enzyme (IDE). The Journal of biological chemistry 286: 37446-37457.
Kim J, Miller V M, Levites Y, West K J, Zwizinski C W, Moore B D, Troendle F J, Bann M, Verbeeck C, Price R W, Smithson L, Sonoda L, Wagg K, Rangachari V, Zou F, Younkin S G, Graff-Radford N, Dickson D, Rosenberry T & Golde T E (2008) BRI2 (ITM2b) inhibits Abeta deposition in vivo. The Journal of neuroscience: the official journal of the Society for Neuroscience 28: 6030-6.
Kim S H, Wang R, Gordon D J, Bass J, Steiner D F, Lynn D G, Thinakaran G, Meredith S C & Sisodia S S (1999) Furin mediates enhanced production of fibrillogenic ABri peptides in familial British dementia. Nature neuroscience 2: 984-8.
Kim S H, Wang R, Gordon D J, Bass J, Steiner D F, Thinakaran G, Lynn D G, Meredith S C & Sisodia S S (2000) Familial British dementia: expression and metabolism of BRI. Annals of the New York Academy of Sciences 920: 93-9.
Korth C, Stierli B, Streit P, Moser M, Schaller O, Fischer R, Schulz-Schaeffer W, Kretzschmar H, Raeber A, Braun U, Ehrensperger F, Hornemann S, Glockshuber R, Riek R, Billeter M, Wüthrich K & Oesch B (1997) Prion (PrPSc)-specific epitope defined by a monoclonal antibody. Nature 390: 74-7.
Kuhn P-H, Wang H, Dislich B, Colombo A, Zeitschel U, Ellwart J W, Kremmer E, Rossner S & Lichtenthaler S F (2010) ADAM10 is the physiologically relevant, constitutive alpha-secretase of the amyloid precursor protein in primary neurons. The EMBO journal 29: 3020-32.
Lashley T, Revesz T, Plant G, Bandopadhyay R, Lees A, Frangione B, Wood N, De Silva R, Ghiso J, Rostagno A & Holton J (2008) Expression of BRI2 mRNA and protein in normal human brain and familial British dementia: its relevance to the pathogenesis of disease. Neuropathology and applied neurobiology 34: 492-505.
Marcinkiewicz M & Seidah N G (2000) Coordinated expression of beta-amyloid precursor protein and the putative β-secretase BACE and alpha-secretase ADAM10 in mouse and human brain. Journal of Neurochemistry 75: 2133-2143.
Martin L, Fluhrer R & Haass C (2009) Substrate requirements for SPPL2b-dependent regulated intramembrane proteolysis. The Journal of biological chemistry 284: 5662-70.
Martin L, Fluhrer R, Reiss K, Kremmer E, Saftig P & Haass C (2008) Regulated intramembrane proteolysis of Bri2 (Itm2b) by ADAM10 and SPPL2a/SPPL2b. The Journal of biological chemistry 283: 1644-52.
Matsuda S, Giliberto L, Matsuda Y, Davies P, McGowan E, Pickford F, Ghiso J, Frangione B & D'Adamio L (2005) The familial dementia BRI2 gene binds the Alzheimer gene amyloid-beta precursor protein and inhibits amyloid-beta production. The Journal of biological chemistry 280: 28912-6.
Matsuda S, Giliberto L, Matsuda Y, McGowan E M & D'Adamio L (2008) BRI2 inhibits amyloid beta-peptide precursor protein processing by interfering with the docking of secretases to the substrate. The Journal of neuroscience: the official journal of the Society for Neuroscience 28: 8668-76.
Matsuda S, Matsuda Y, Snapp E L & D'Adamio L (2009) Maturation of BRI2 generates a specific inhibitor that reduces APP processing at the plasma membrane and in endocytic vesicles. Neurobiology of aging 32: 1400-8.
Matsuda S, Tamayev R & D'Adamio L (2011) Increased AβPP Processing in Familial Danish Dementia Patients. Journal of Alzheimer's disease: JAD.
Mulder C, Verwey N A, Van der Flier W M, Bouwman F H, Kok A, Van Elk E J, Scheltens P & Blankenstein M a (2010) Amyloid-beta(1-42), total tau, and phosphorylated tau as cerebrospinal fluid biomarkers for the diagnosis of Alzheimer disease. Clinical chemistry 56: 248-53.
Nelson P T, Jicha G A, Schmitt F A, Liu H, Davis D G, Mendiondo M S, Abner E L & Markesbery W R (2007) Clinicopathologic correlations in a large Alzheimer disease center autopsy cohort: neuritic plaques and neurofibrillary tangles “do count” when staging disease severity. Journal of neuropathology and experimental neurology 66: 1136-46.
Peng S, Fitzen M, Jörnvall H & Johansson J (2010) The extracellular domain of Bri2 (ITM2B) binds the ABri peptide (1-23) and amyloid beta-peptide (Abeta1-40): Implications for Bri2 effects on processing of amyloid precursor protein and Abeta aggregation. Biochemical and biophysical research communications 393: 356-61.
Pham T V, Piersma S R, Warmoes M & Jimenez C R (2010) On the beta-binomial model for analysis of spectral count data in label-free tandem mass spectrometry-based proteomics. Bioinformatics (Oxford, England) 26: 363-9.
Reisberg B, Ferris S., De Leon M., Ed D & Crook T (1982) The Global Deterioration Scale for Assessment of Primary degenerative Dementia. Am J Psychiatry 139: 1136-39.
Rostagno A, Tomidokoro Y, Lashley T, Ng D, Plant G, Holton J, Frangione B, Revesz T & Ghiso J (2005) Chromosome 13 dementias. Cellular and molecular life sciences: CMLS 62: 1814-25.
Tamayev R & D'Adamio L (2012) Memory deficits of British Dementia knock-in mice are prevented by APP haploinsufficiency. The Journal of neuroscience: the official journal of the Society for Neuroscience 32: 5481-5485.
Tamayev R, Giliberto L, Li W, D'Abramo C, Arancio O, Vidal R & D'Adamio L (2010a) Memory Deficits Due to Familial British Dementia BRI2 Mutation Are Caused by Loss of BRI2 Function Rather than Amyloidosis. The Journal of neuroscience: the official journal of the Society for Neuroscience 30: 14915-14924.
Tamayev R, Matsuda S, Arancio O & D'Adamio L (2012) β- but not γ-secretase proteolysis of APP causes synaptic and memory deficits in a mouse model of dementia. EMBO molecular medicine 4: 171-9.
Tamayev R, Matsuda S, Fa M, Arancio O & Adamio L D (2010b) Danish dementia mice suggest that loss of function and not the amyloid cascade causes synaptic plasticity and memory deficits. PNAS 107: 20822-27.
Tamayev R, Matsuda S, Giliberto L, Arancio O & D'Adamio L (2011) APP heterozygosity averts memory deficit in knockin mice expressing the Danish dementia BRI2 mutant. The EMBO journal 30: 2501-9.
Teunissen C, Petzold A, Bennett J, Berven F, Brundin L, Comabella M, Franciotta D, Frederiksen J, Fleming J, Furlan R, Hintzen R, Hughes S, Johnson M, Krasulova E, Kuhle J, Magnone M, Rajda C, Rejdak K, Schmidt H, Van Pesch V, et al. (2009) A consensus protocol for the standardization of cerebrospinal fluid collection and biobanking. Neurology 73: 1914-22.
Teunissen C E, Tumani H, Bennett J L, Berven F S, Brundin L, Comabella M, Franciotta D, Federiksen J L, Fleming J O, Furlan R, Hintzen R Q, Hughes S G, Jimenez C R, Johnson M H, Killestein J, Krasulova E, Kuhle J, Magnone M-C, Petzold A, Rajda C, et al. (2011) Consensus Guidelines for CSF and Blood Biobanking for CNS Biomarker Studies. Multiple sclerosis international 2011: 246412.
Thal D R, Capetillo-Zarate E, Del Tredici K & Braak H (2006) The development of amyloid beta protein deposits in the aged brain. Science of aging knowledge environment: SAGE KE 6: 1-9.
Tsachaki M, Ghiso J & Efthimiopoulos S (2008) BRI2 as a central protein involved in neurodegeneration. Biotechnology journal 3: 1548-54.
Tsachaki M, Ghiso J, Rostagno A & Efthimiopoulos S (2010) BRI2 homodimerizes with the involvement of intermolecular disulfide bonds. Neurobiology of aging 31: 88-98.
Tsachaki M, Serlidaki D, Fetani A, Zarkou V, Rozani I, Ghiso J & Efthimiopoulos S (2011) Glycosylation of BRI2 on asparagine 170 is involved in its trafficking to the cell surface but not in its processing by furin or ADAM10. Glycobiology 21: 1382-8.
Vidal R, Frangione B, Rostagno A, Mead S, Révész T, Plant G & Ghiso J (1999) A stop-codon mutation in the BRI gene associated with familial British dementia. Nature 399: 776-81.
Vidal R, Revesz T, Rostagno A, Kim E, Holton J L, Bek T, Bojsen-Møller M, Braendgaard H, Plant G, Ghiso J & Frangione B (2000) A decamer duplication in the 3′ region of the BRI gene originates an amyloid peptide that is associated with dementia in a Danish kindred. Proceedings of the National Academy of Sciences of the United States of America 97: 4920-5.
Willander H, Hermansson E, Johansson J & Presto J (2011) BRICHOS domain associated with lung fibrosis, dementia and cancer—a chaperone that prevents amyloid fibril formation? The FEBS journal 278: 3893-904.
Willander H, Presto J, Askarieh G, Biverstal H, Frohm B, Knight S D, Johansson J & Linse S (2012) BRICHOS Domains Efficiently Delay Fibrillation of Amyloid β-Peptide. The Journal of biological chemistry 287: 31608-17.
Yamaguchi H, Haga C, Hirai S, Nakazato Y & Kosaka K (1990) Distinctive, rapid, and easy labelling of diffuse plaques in the Alzheimer brains by a new methenamine silver stain. Acta neuropathologica 79: 569-72.

TABLE 1
Demographic data of patients used in this study.

| | | | | | | Grade | Grade |
|---|---|---|---|---|---|---|---|
| | Clinical | Pathological | | | | (Braak, | (Thal, |
| Patient number | diagnosis | diagnosis | Gender | Age | PMI (h) | NFT) | Aβ) |
| 1 | Control | Control | M | 56 | 5.50 | 0 | 0 |
| 2 | Control | Control | M | 80 | 10.00 | 0 | 0 |
| 3 | Control | Control | M | 56 | 5.50 | 0 | 4 |
| 4 | Control | Control | M | 66 | 9.15 | 0 | 1 |
| 5 | Control | Control | M | 96 | 6.30 | I | 0 |
| 6 | Control | Control | F | 84 | 6.55 | I | 0 |
| 7 | Control | Control | F | 94 | 6.25 | I | 0 |
| 8 | Control | Control | F | 77 | 2.55 | I | 1 |
| 9 | Control | Control | F | 73 | 7.45 | I | 3 |
| 10 | Control | Control | M | 81 | 5.30 | II | 0 |
| 11 | Control | Control | F | 93 | 7.15 | II | 0 |
| 12 | Control | Control | M | 88 | 4.43 | II | 1 |
| 13 | Control | Control | M | 71 | 8.55 | II | 3 |
| 14 | Control | Control | F | 89 | 6.05 | II | 3 |
| 15 | Control | Control | F | 89 | 3.52 | III | 0 |
| 16 | Control | Control | M | 88 | 7.00 | III | 1 |
| 17 | Control | Control | M | 74 | 5.00 | III | 3 |
| 18 | Control | Uncertain | F | 86 | 6.25 | III | 1 |
| 19 | AD | Uncertain | F | 83 | 4.05 | III | 2 |
| 20 | AD | Uncertain | M | 82 | 5.20 | III | 0 |
| 21 | AD | AD | F | 86 | 7.45 | IV | 4 |
| 22 | AD | AD | F | 93 | 2.30 | IV | ND |
| 23 | AD | AD | M | 93 | 5.50 | IV | 4 |
| 24 | AD | AD | M | 61 | 5.55 | V | 4 |
| 25 | AD | AD | F | 81 | ND | V | 4 |
| 26 | AD | AD | F | 78 | 4.50 | V | 4 |
| 27 | AD | AD | M | 93 | 4.30 | V | 4 |
| 28 | AD | AD | M | 74 | 5.35 | VI | 4 |
| 29 | AD | AD | F | 72 | 5.55 | VI | 4 |
| 30 | AD | AD | F | 68 | 3.50 | VI | 4 |
| 31 | AD | AD | F | 67 | 5.50 | VI | 4 |
| 32 | AD | AD | F | 91 | 5.45 | VI | ND |
| 33 | VD | AD | F | 92 | 4.00 | IV | 4 |
| 34 | VD | AD | F | 91 | 4.15 | IV | 4 |
| 35 | VD | AD | F | 78 | 4.35 | V | 4 |
| 36 | NA | PiD | M | 70 | 5.15 | NA | NA |
| 37 | NA | PD with | M | 82 | 19 | IV | NA |
| | | dementia | | | | | |
| 38 | NA | GSS | M | 52 | 5.45 | NA | NA |
| 39 | NA | GSS | F | 45 | NA | NA | NA |
| 40 | NA | FTD (mut. | M | 46 | 5.35 | NA | NA |
| | | P301L) | | | | | |

Braak and Thal stages were established as described in the Materials and Methods section.
Abbreviations:
AD = Alzheimer's disease;
PiD = Pick's disease (Type A);
PD = Parkinson's disease;
GSS = Gerstmann-Straussler-Scheinker syndrome (Prion disease);
F = Female;
M = Male;
PMI = Post-mortem interval (h = hours);
VD = Vascular dementia;
ND = Not determined;
NA = Not applicable.

TABLE II
Demographic data of CSF samples.

| | | | | | | Total | | |
|---|---|---|---|---|---|---|---|---|
| | | | | | | Protein | | MMSE |
| Patient | Age | No | Aβ₁₋₄₂ | t-tau | p-tau | concentration | MMSE | follow-up |
| Groups | (mean ± SD) | (M/F) | (pg/mL) | (pg/mL) | (pg/mL) | (μg/μL) | (baseline) | (mean ± SD) |
| SMC | 60.3 ± 4.5 | 4(2/2) | 838 ± 133 | 200 ± 76 | 47 ± 16 | 0.34 ± 0.11 | 29.5 ± 1.0. | / |
| MCI-S | 62.1 ± 3.2 | 4(1/3) | 875 ± 201 | 421 ± 347 | 73 ± 48 | 0.43 ± 0.24 | 27.4 ± 2.2 | 28.4 ± 2.0 |
| MCI-AD | 66.2 ± 6.4 | 5(2/3) | 499 ± 78 | 1071 ± 248^{a} | 138 ± 37^{a} | 0.31 ± 0.09 | 27.0 ± 1.4 | 25.0 ± 2.5 |
| AD | 63.9 ± 6.6 | 5(2/3) | 384 ± 146^{a,b} | 526 ± 120 | 102 ± 40 | 0.46 ± 0.17 | 21.4 ± 6.3 | 20.8 ± 5.6 |

Data are reported as medians and 25-75% percentiles unless indicated.
SMC = subjective memory complaints,
MCI-S = MCI with stable disease,
MCI-AD = MCI converting to AD,
AD = probable AD.
^{a}= at least p < 0.05 from SMC,
^{b}= at least p < 0.05 from MCI-S.

1. A method for determining the level of BRI2 polypeptide in an individual, the method comprising:
contacting a sample from the individual with a BRI2 binding compound.

2. The method according to claim 1, wherein the contacting occurs in vitro.

3. A method of determining the risk of developing Alzheimer's disease in an individual, the method comprising determining the level of BRI2 polypeptide in the individual and comparing the level of BRI2 to a reference value.

4. The method according to claim 3, wherein a BRI2 level higher than the reference value indicates a risk of developing Alzheimer's disease.

5. The method according to claim 1, wherein the level of BRI2 is determined in vitro from a sample from the individual.

6. The method according to claim 1, wherein the sample is cerebral spinal fluid or blood.

7. The method according to claim 3, wherein the level of BRI2 is determined with a BRI2 binding compound.

8. The method according to claim 1, wherein the binding compound binds to amino acids 137-231 of SEQ ID NO:1.

9. The method according to claim 8, wherein the binding compound binds to amino acids 140-153 of SEQ ID NO:1.

10. The method according to claim 1, wherein the binding compound is a monoclonal antibody.

11. The method according to claim 1, wherein the binding compound is a polyclonal antibody which does not bind to SEQ ID NO:1 at amino acids outside of 140-153.

12. The method according to claim 1, wherein the level of BRI2 is determined in vivo.

13. The method according to claim 12, wherein the level of BRI2 is determined in the hippocampus of the individual.

14. The method according to claim 12, comprising:
(a) administering to an individual a positron emission tomography (PET)-compatible tracer that binds to BRI2;
(b) carrying out a PET scan of the individual; and
(c) deter mining the signal intensity of the tracer.

15. The method according to claim 14, wherein a tracer intensity higher than a reference value indicates a risk of developing Alzheimer's disease.

16. A method for identifying compounds for treating Alzheimer's disease during preclinical stages, the method comprising:
(a) administering one or more candidate compounds to a preclinical animal model of Alzheimer's disease;
(b) assessing changes in BRI2 in the animal model relative to measures of BRI2 in a control animal; and
(c) selecting a candidate compound that induces a change in BRI2 toward measures of BRI2 in a control animal.

17. A method for treating Alzheimer's disease in an individual in need thereof, the method comprising administering to the individual a therapeutically effective amount of a compound that reduces the level of BRI2 protein.

18. The method according to claim 17, wherein the compound is a BRI2 binding molecule.

19. The method according to claim 18, wherein the binding molecule binds to amino acids 137-231 of SEQ ID NO:1.

20. The method according to claim 19, wherein the binding molecule binds to amino acids 140-153 of SEQ ID NO:1.

21. The method according to claim 17, wherein the compound reduces the level of abnormal or non-functional BRI2 protein.

22. An anti-BRI2 antibody that binds to amino acids 140-153 of SEQ ID NO:1.

23. The antibody of claim 22, wherein the antibody is a monoclonal antibody.

24. The antibody of claim 22, wherein the antibody is polyclonal and does not bind to SEQ ID NO:1 at amino acids outside of 140-153.

25. A polynucleotide encoding the antibody of claim 22.

26. A vector comprising at least one polynucleotide of claim 25.

27. A method of determining the risk of developing Alzheimer's disease in an individual, the method comprising:
utilizing the antibody of claim 22 for determining the risk of developing Alzheimer's disease in an individual.

28. A method of treating a subject believed to be suffering from Alzheimer's disease, the method comprising:
utilizing the antibody of claim 22 in the treatment of the subject.

29. A method for treating Alzheimer's disease in an individual, the method comprising:
(a) determining the level of BRI2 polypeptide in the individual,
(b) comparing the level of BRI2 to a reference value; and
(c) treating an individual having an altered level of BRI2 over the reference value with an Alzheimer's disease treatment.


SEQUENCE LISTING (1 sequence)

SEQ ID NO: 1 - protein, 266 aa, Homo sapiens
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS